(19) United States
(12) Patent Application Publication
(10) Pub. No.: US 2024/0347679 A1
(43) Pub. Date: Oct. 17, 2024

(54) LIGHT GENERATING SYSTEM PROVIDING UV LIGHT

(71) Applicant: SIGNIFY HOLDING B.V., EINDHOVEN (NL)

(72) Inventors: TIES VAN BOMMEL, HORST (NL); RIFAT ATA MUSTAFA HIKMET, EINDHOVEN (NL)

(21) Appl. No.: 18/294,614

(22) PCT Filed: Jul. 18, 2022
(86) PCT No.: PCT/EP2022/069985

§ 371 Date: Feb. 2, 2024

(30) Foreign Application Priority Data
Aug. 2, 2021 (EP) 21189019.9

(51) Int. Cl. H01L 33/50; A61L 2/10; A61L 9/20; H01L 33/48

(52) U.S. Cl. CPC H01L 33/507; A61L 2/10; A61L 9/20; H01L 33/483; A61L 2209/12

(57) ABSTRACT

The invention provides a light generating system (1000) comprising (i) a first light generating device (110), an optical element (500), and a first converter material (210), wherein: (A) the first light generating device (110) is configured to generate first device light (111), wherein the first device light (111) comprises one or more of visible light and infrared radiation: (B) the optical element (500) is configured in a light receiving relationship with the first light generating device (110); wherein the optical element (500) is transmissive for the first device light (111): (C) the first converter material (210) is configured downstream of the optical element (500); wherein the first converter material (210) is configured to convert at least part of the first device light (111) transmitted by the optical element (500) into first converter material light (211): wherein the first light generating device (110) and the first converter material (210) are selected such that the first converter material light (211) has spectral power at one or more wavelengths in the wavelength range of smaller than 380 nm; and (D) wherein the optical element (500) has a lower transmission for the first converter material light (211) than for the first device light (111).

### FIELD OF THE INVENTION

The invention relates to a light generating system and to a lighting device comprising such light generating system. The invention also relates to a method for treating a gas or a surface.

### BACKGROUND OF THE INVENTION

Antimicrobial upconversion systems are known in the art. US2010/0297206, for instance, describes antimicrobial articles, systems, and methods for killing, inactivating, and/or inhibiting microorganisms. The antimicrobial articles and systems utilize up-conversion luminescence wherein a phosphor or luminescent material is capable of absorbing visible, infrared light, or longer wavelength radiation and emitting antimicrobial ultraviolet radiation via upconversion thus inhibiting the growth of, inhibiting the reproduction of or killing or otherwise inactivating microorganisms such as, but not limited to, spores, bacteria, fungi, mildew, mold, and algae. Embodiments of the antimicrobial article or system may comprise such a luminescent material and thus will have antimicrobial activity when exposed to natural or artificial light.

### SUMMARY OF THE INVENTION

UV light has been used for disinfection for over 100 years. Wavelengths between about 190 nm and 300 nm may be strongly absorbed by nucleic acids, which may result in defects in an organism's genome. This may be desired for inactivating (killing), bacteria and viruses, but may also have undesired side effects for humans. Therefore, the selection of wavelength of radiation, intensity of radiation and duration of irradiation may be limited in environments where people may reside such as offices, public transport, cinema's, restaurants, shops, etc., thus limiting the disinfection capacity. Especially in such environments, additional measures of disinfection may be advantageous to prevent the spread of bacteria and viruses such as influenza or novel (corona) viruses like COVID-19, SARS and MERS.

It appears desirable to produce systems, that provide alternative ways for air treatment, such as disinfection. Further, existing systems for disinfection may not easily be implemented in existing infrastructure, such as in existing buildings like offices, hospitality areas, etc. and/or may not easily be able to serve larger spaces. This may again increase the risk of contamination. Further, incorporation in HVAC systems may not lead to desirable effects and appears to be relatively complex. Further, existing systems may not be efficient, or may be relatively bulky, and may also not easily be incorporated in functional devices, such as e.g. luminaires.

Other disinfection systems may use one or more anti-microbial and/or anti-viral means to disinfect a space or an object. Examples of such means may be chemical agents which may raise concerns. For instance, the chemical agents may also be harmful for people and pets.

In embodiments, the disinfecting light, may especially comprise ultraviolet (UV) radiation (and/or optionally violet radiation), i.e., the light may comprise a wavelength selected from the ultraviolet wavelength range (and/or optionally the violet wavelength range). However, other wavelengths are herein not excluded. The ultraviolet wavelength range is defined as light in a wavelength range from 100 to 380 nm and can be divided into different types of UV light/UV wavelength ranges (Table 1). Different UV wavelengths of radiation may have different properties and thus may have different compatibility with human presence and may have different effects when used for disinfection (Table 1).

TABLE 1
Properties of different types of UV, violet, and NIR wavelength light

| | | | (Relative) | | | | |
|---|---|---|---|---|---|---|---|
| | | | sterilization | | | | |
| | Short | Wavelength | effectiveness | | Safe | Vitamin D | Ozone |
| Name | name | (nm) | Bacteria | Viruses | Radiation | generation | generation |
| Near | NIR | 780-950 | + | +/− | +++ | | |
| Infrared | | | | | | | |
| (deep red) | | | | | | | |
| Violet | V | 380-420 | +/− | − | + | | |
| Ultra- | UV-A | 315-380 | + | − | + | | |
| violet A | | | | | | | |
| Ultra- | UV-B | 280-315 | + | +/− | +/− | + | |
| violet B | | | | | | | |
| Near | Near | 230-280 | ++ | ++ | − | | |
| ultraviolet | UV-C | | | | | | |
| C | | | | | | | |
| Far ultra- | Far UV- | 190-230 | +++ | +++ | + | | +/− |
| violet C | C | | | | | | |
| Extreme | Extreme | 100-190 | +++ | +++ | − | | + |
| ultraviolet | UV-C | | | | | | |
| C | | | | | | | |

Each UV type/wavelength range may have different benefits and/or drawbacks. Relevant aspects may be (relative) sterilization effectiveness, safety (regarding radiation), and ozone production (as result of its radiation). Depending on an application a specific type of UV light or a specific combination of UV light types may be selected and provides superior performance over other types of UV light. UV-A may be (relatively) safe and may inactivate (kill) bacteria, but may be less effective in inactivating (killing) viruses. UV-B may be (relatively) safe when a low dose (i.e. low exposure time and/or low intensity) is used, may inactivate (kill) bacteria, and may be moderately effective in inactivating (killing) viruses. UV-B may also have the additional benefit that it can be used effectively in the production of vitamin D in a skin of a person or animal. Near UV-C may be relatively unsafe, but may effectively inactivating, especially kill bacteria and viruses. Far UV-C may also be effective in inactivating (killing) bacteria and viruses, but may be (relatively to other UV-C wavelength ranges) (rather) safe. Far UV-C light may generate some ozone which may be harmful for human beings and animals. Extreme UV-C may also be effective in inactivating (killing) bacteria and viruses, but may be relatively unsafe. Extreme UV-C may generate ozone which may be undesired when exposed to human beings or animals. In some application ozone may be desired and may contribute to disinfection, but then its shielding from humans and animals may be desired. Hence, in the table “+” for ozone production especially implies that ozone is produced which may be useful for disinfection applications, but may be harmful for humans/animals when they are exposed to it. Hence, in many applications this “+” may actually be undesired while in others, it may be desired. The types of light indicated in above table may in embodiments be used to sanitize air and/or surfaces.

The terms “inactivating” and “killing” with respect to a virus may herein especially refer to damaging the virus in such a way that the virus can no longer infect and/or reproduce in a host cell, i.e., the virus may be (essentially) harmless after inactivation or killing.

Hence, in embodiments, the light may comprise a wavelength in the UV-A range. In further embodiments, the light may comprise a wavelength in the UV-B range. In further embodiments, the light may comprise a wavelength in the Near UV-C range. In further embodiments, the light may comprise a wavelength in the Far UV-C range. In further embodiments, the light may comprise a wavelength in the extreme UV-C range. The Near UV-C, the Far UV-C and the extreme UV-C ranges may herein also collectively be referred to as the UV-C range. Hence, in embodiments, the light may comprise a wavelength in the UV-C range. In other embodiments, the light may comprise violet radiation.

It appears desirable to implement disinfection devices in existing infrastructures. This may save space and may also allow a kind of intuitive use of disinfection devices. Especially, it appears to be a desire to provide disinfection devices not as additional device but integrated these in other systems or devices. Further, there appears to be a desire, also in view of safety, to provide disinfection devices in a way that a substantial area of a space can be disinfected, and not only part of a space.

Hence, it is an aspect of the invention to provide an alternative radiation based disinfection system (or device), which preferably further at least partly obviates one or more of above-described draw backs. The present invention may have as object to overcome or ameliorate at least one of the disadvantages of the prior art, or to provide a useful alternative.

In a first aspect, the invention provides light generating system (“system”) comprising (i) a first light generating device, an optical element, and a first converter material. In embodiments, the first light generating device may especially be configured to generate first device light. In embodiments, the first device light may comprise one or more of visible light and infrared radiation (“infrared light”). The optical element may especially be configured in a light receiving relationship with the first light generating device. Further, in embodiments the optical element may be transmissive for the first device light. Especially, the first converter material (“converter material”) may be configured downstream of the optical element. In embodiments, the first converter material may be configured to convert at least part of the first device light transmitted by the optical element into first converter material light. Especially, in embodiments the first light generating device and the first converter material may be selected such that the first converter material light has spectral power at one or more wavelengths in the wavelength range of smaller than 380 nm. Yet, in specific embodiments the optical element has a lower transmission for the first converter material light than for the first device light. Hence, especially the invention provides in embodiments a light generating system comprising (i) a first light generating device, an optical element, and a first converter material, wherein: (A) the first light generating device is configured to generate first device light, wherein the first device light comprises one or more of visible light and infrared radiation: (B) the optical element is configured in a light receiving relationship with the first light generating device: wherein the optical element is transmissive for the first device light: (C) the first converter material is configured downstream of the optical element: wherein the first converter material is configured to convert at least part of the first device light transmitted by the optical element into first converter material light: wherein the first light generating device and the first converter material are selected such that the first converter material light has spectral power at one or more wavelengths in the wavelength range of smaller than 380 nm; and (D) wherein the optical element has a lower transmission for the first converter material light than for the first device light.

Such system may be integrated in existing infrastructures, such as existing lighting grids (like grids of luminaires). This may save space and may also allow a kind of intuitive use of disinfection devices. This may also allow disinfecting a substantial area of a space instead of only parts of a space. Further, an (space) efficient way a lighting device may be provided that may also have a disinfection function. Amongst others, the invention allows (in embodiments) solid-state UV light disinfection. However, with the invention it is not necessary to use a UV generating device, like a UV radiation generating solid state light source. With the invention, it is possible to generate disinfection radiation (in the UV) while using a (solid state) light source configured to provide radiation in the visible and/or infrared.

As indicated above, the light generating system may comprise a first light generating device. The term “first light generating device” may also refer to a plurality of (different) first light generating devices. Further, optionally the light generating system may comprise a second light generating device. The term “second light generating device” may also refer to a plurality of (different) second light generating devices. The term “light generating device” may refer to one or more light generating devices. Each light generating device may comprise one or more light sources, especially one or more solid state light sources.

The first light generating device may comprise one or more light sources, especially one or more solid state light sources. The second light generating device may comprise one or more light sources, especially one or more solid state light sources. Especially, in embodiments the first light generating device and the second light generating device have at least operational modes wherein the spectral power distributions of the light generated by the first light generating device and the second light generating device are different. The first light generating device may be configured to provide first device light and the second light generating device may be configured to generate second device light. In specific embodiments, the first device light and the second device light differ in spectral power distribution, though this is not necessarily the case in all embodiments. Hence, in specific embodiments the first device light and the second device light may differ in color point. In specific embodiments, colors or color points of a first type of light and a second type of light may be different when the respective color points of the first type of light and the second type of light differ with at least 0.01 for u′ and/or with at least 0.01 for v′, even more especially at least 0.02 for u′ and/or with at least 0.02 for v′. In yet more specific embodiments, the respective color points of first type of light and the second type of light may differ with at least 0.03 for u′ and/or with at least 0.03 for v′. Here, u′ and v′ are color coordinate of the light in the CIE 1976 UCS (uniform chromaticity scale) diagram. Note that the second light generating device(s) is (are) optional.

The term “light source” may in principle relate to any light source known in the art. It may be a conventional (tungsten) light bulb, a low pressure mercury lamp, a high pressure mercury lamp, a fluorescent lamp, a LED (light emissive diode). In a specific embodiment, the light source comprises a solid state LED light source (such as a LED or laser diode (or “diode laser”)). The term “light source” may also relate to a plurality of light sources, such as 2-200 (solid state) LED light sources. Hence, the term LED may also refer to a plurality of LEDs. Further, the term “light source” may in embodiments also refer to a so-called chips-on-board (COB) light source. The term “COB” especially refers to LED chips in the form of a semiconductor chip that is neither encased nor connected but directly mounted onto a substrate, such as a PCB. Hence, a plurality of light semiconductor light source may be configured on the same substrate. In embodiments, a COB is a multi LED chip configured together as a single lighting module.

The light source has a light escape surface. Referring to conventional light sources such as light bulbs or fluorescent lamps, it may be outer surface of the glass or quartz envelope. For LED's it may for instance be the LED die, or when a resin is applied to the LED die, the outer surface of the resin. In principle, it may also be the terminal end of a fiber. The term escape surface especially relates to that part of the light source, where the light actually leaves or escapes from the light source. The light source is configured to provide a beam of light. This beam of light (thus) escapes from the light exit surface of the light source.

The term “light source” may refer to a semiconductor light-emitting device, such as a light emitting diode (LEDs), a resonant cavity light emitting diode (RCLED), a vertical cavity laser diode (VCSELs), an edge emitting laser, etc. . . . . The term “light source” may also refer to an organic light-emitting diode, such as a passive-matrix (PMOLED) or an active-matrix (AMOLED). In a specific embodiment, the light source comprises a solid-state light source (such as a LED or laser diode). In an embodiment, the light source comprises a LED (light emitting diode). The terms “light source” or “solid state light source” may also refer to a superluminescent diode (SLED).

The term LED may also refer to a plurality of LEDs. Further, the term “light source” may in embodiments also refer to a so-called chips-on-board (COB) light source. The term “COB” especially refers to LED chips in the form of a semiconductor chip that is neither encased nor connected but directly mounted onto a substrate, such as a PCB. Hence, a plurality of semiconductor light sources may be configured on the same substrate. In embodiments, a COB is a multi-LED chip configured together as a single lighting module.

The term “light source” may also relate to a plurality of (essentially identical (or different)) light sources, such as 2-2000 solid state light sources. In embodiments, the light source may comprise one or more micro-optical elements (array of micro lenses) downstream of a single solid-state light source, such as a LED, or downstream of a plurality of solid-state light sources (i.e. e.g. shared by multiple LEDs). In embodiments, the light source may comprise a LED with on-chip optics. In embodiments, the light source comprises a pixelated single LEDs (with or without optics) (offering in embodiments on-chip beam steering).

In embodiments, the light source may be configured to provide primary radiation, which is used as such, such as e.g. a blue light source, like a blue LED, or a green light source, such as a green LED, and a red light source, such as a red LED. Such LEDs, which may not comprise a luminescent material (“phosphor”) may be indicated as direct color LEDs.

In other embodiments, however, the light source may be configured to provide primary radiation and part of the primary radiation is converted into secondary radiation. Secondary radiation may be based on conversion by a luminescent material. The secondary radiation may therefore also be indicated as luminescent material radiation. The luminescent material may in embodiments be comprised by the light source, such as a LED with a luminescent material layer or dome comprising luminescent material. Such LEDs may be indicated as phosphor converted LEDs or PC LEDs (phosphor converted LEDs). In other embodiments, the luminescent material may be configured at some distance (“remote”) from the light source, such as a LED with a luminescent material layer not in physical contact with a die of the LED. Hence, in specific embodiments the light source may be a light source that during operation emits at least light at wavelength selected from the range of 380-470 nm. However, other wavelengths may also be possible. This light may partially be used by the luminescent material.

In embodiments, the light generating device may comprise a luminescent material. In embodiments, the light generating device may comprise a PC LED. In other embodiments, the light generating device may comprise a direct LED (i.e. no phosphor). In embodiments, the light generating device may comprise a laser device, like a laser diode. In embodiments, the light generating device may comprise a superluminescent diode. Hence, in specific embodiments, the light source may be selected from the group of laser diodes and superluminescent diodes. In other embodiments, the light source may comprise an LED.

The light source may especially be configured to generate light source light having an optical axis (O), (a beam shape) and a spectral power distribution. The light source light may in embodiments comprise one or more bands, having band widths as known for lasers

The term “light source” may (thus) refer to a light generating element as such, like e.g. a solid state light source, or e.g. to a package of the light generating element, such as a solid state light source, and one or more of a luminescent material comprising element and (other) optics, like a lens, a collimator. A light converter element (“converter element” or “converter”) may comprise a luminescent material comprising element. For instance, a solid state light as such, like a blue LED, is a light source. A combination of a solid state light source (as light generating element) and a light converter element, such as a blue LED and a light converter element, optically coupled to the solid state light source, may also be a light source. Hence, a white LED is a light source.

The term “light source” herein may also refer to a light source comprising a solid state light source, such as an LED or a laser diode or a superluminescent diode. The “term light source” may (thus) in embodiments also refer to a light source that is (also) based on conversion of light, such as a light source in combination with a luminescent converter material. Hence, the term “light source” may also refer to a combination of a LED with a luminescent material configured to convert at least part of the LED radiation, or to a combination of a (diode) laser with a luminescent material configured to convert at least part of the (diode) laser radiation.

The phrases “different light sources” or “a plurality of different light sources”, and similar phrases, may in embodiments refer to a plurality of solid-state light sources selected from at least two different bins. Likewise, the phrases “identical light sources” or “a plurality of same light sources”, and similar phrases, may in embodiments refer to a plurality of solid-state light sources selected from the same bin.

In specific embodiments, the light generating device may comprise a plurality of different light sources, such as two or more subsets of light sources, with each subset comprising one or more light sources configured to generate light source light having essentially the same spectral power distribution, but wherein light sources of different subsets are configured to generate light source light having different spectral distributions. In such embodiments, a control system may be configured to control the plurality of light sources. In specific embodiments, the control system may control the subsets of light sources individually.

The term “laser light source” especially refers to a laser. Such laser may especially be configured to generate laser light source light having one or more wavelengths in the UV, visible, or infrared, especially having a wavelength selected from the spectral wavelength range of 200-2000 nm, such as 300-1500 nm. The term “laser” especially refers to a device that emits light through a process of optical amplification based on the stimulated emission of electromagnetic radiation.

Especially, in embodiments the term “laser” may refer to a solid-state laser. In specific embodiments, the terms “laser” or “laser light source”, or similar terms, refer to a laser diode (or diode laser).

Hence, in embodiments the light source comprises a laser light source. In embodiments, the terms “laser” or “solid state laser” may refer to one or more of cerium doped lithium strontium (or calcium) aluminum fluoride (Ce:LiSAF, Ce:LiCAF), chromium doped chrysoberyl (alexandrite) laser, chromium ZnSe (Cr:ZnSe) laser, divalent samarium doped calcium fluoride (Sm:CaF₂) laser, Er:YAG laser, erbium doped and erbium-ytterbium codoped glass lasers, F-Center laser, holmium YAG (Ho:YAG) laser, Nd:YAG laser, NdCrYAG laser, neodymium doped yttrium calcium oxoborate Nd:YCaIO(BO₃)₃ or Nd:YCOB, neodymium doped yttrium orthovanadate (Nd:YVO₄) laser, neodymium glass (Nd:glass) laser, neodymium YLF (Nd:YLF) solid-state laser, promethium 147 doped phosphate glass (147Pm³⁺:glass) solid-state laser, ruby laser (Al₂O₃:Cr³⁺), thulium YAG (Tm:YAG) laser, titanium sapphire (Ti:sapphire; Al₂O₃:Ti³⁺) laser, trivalent uranium doped calcium fluoride (U:CaF₂) solid-state laser, Ytterbium doped glass laser (rod, plate/chip, and fiber), Ytterbium YAG (Yb:YAG) laser, Yb₂O₃ (glass or ceramics) laser, etc.

For instance, including second and third harmonic generation embodiments, the light source may comprise one or more of an F center laser, a yttrium orthovanadate (Nd:YVO₄) laser, a promethium 147 doped phosphate glass (147Pm³⁺:glass), and a titanium sapphire (Ti:sapphire; Al₂O₃:Ti³⁺) laser. For instance, considering second and third harmonic generation, such light sources may be used to generated blue light. Further, e.g. an InGaN laser may be applied.

In embodiments, the terms “laser” or “solid state laser” may refer to one or more of a semiconductor laser diodes, such as GaN, InGaN, AlGaInP, AlGaAs, InGaAsP, lead salt, vertical cavity surface emitting laser (VCSEL), quantum cascade laser, hybrid silicon laser, etc.

A laser may be combined with an upconverter in order to arrive at shorter (laser) wavelengths. For instance, with some (trivalent) rare earth ions upconversion may be obtained or with non-linear crystals upconversion can be obtained. Alternatively, a laser can be combined with a downconverter, such as a dye laser, to arrive at longer (laser) wavelengths.

As can be derived from the below, the term “laser light source” may also refer to a plurality of (different or identical) laser light sources. In specific embodiments, the term “laser light source” may refer to a plurality N of (identical) laser light sources. In embodiments, N=2, or more. In specific embodiments, N may be at least 5, such as especially at least 8. In this way, a higher brightness may be obtained. In embodiments, laser light sources may be arranged in a laser bank (see also above). The laser bank may in embodiments comprise heat sinking and/or optics e.g. a lens to collimate the laser light.

The laser light source is configured to generate laser light source light (or “laser light”). The light source light may essentially consist of the laser light source light. The light source light may also comprise laser light source light of two or more (different or identical) laser light sources. For instance, the laser light source light of two or more (different or identical) laser light sources may be coupled into a light guide, to provide a single beam of light comprising the laser light source light of the two or more (different or identical) laser light sources. In specific embodiments, the light source light is thus especially collimated light source light. In yet further embodiments, the light source light is especially (collimated) laser light source light.

The laser light source light may in embodiments comprise one or more bands, having band widths as known for lasers. In specific embodiments, the band(s) may be relatively sharp line(s), such as having full width half maximum (FWHM) in the range of less than 20 nm at RT, such as equal to or less than 10 nm. Hence, the light source light has a spectral power distribution (intensity on an energy scale as function of the wavelength) which may comprise one or more (narrow) bands.

The beams (of light source light) may be focused or collimated beams of (laser) light source light. The term “focused” may especially refer to converging to a small spot. This small spot may be at the discrete converter region, or (slightly) upstream thereof or (slightly) downstream thereof. Especially, focusing and/or collimation may be such that the cross-sectional shape (perpendicular to the optical axis) of the beam at the discrete converter region (at the side face) is essentially not larger than the cross-section shape (perpendicular to the optical axis) of the discrete converter region (where the light source light irradiates the discrete converter region). Focusing may be executed with one or more optics, like (focusing) lenses. Especially, two lenses may be applied to focus the laser light source light. Collimation may be executed with one or more (other) optics, like collimation elements, such as lenses and/or parabolic mirrors. In embodiments, the beam of (laser) light source light may be relatively highly collimated, such as in embodiments ≤2° (FWHM), more especially ≤1° (FWHM), most especially ≤0.5° (FWHM). Hence, ≤2° (FWHM) may be considered (highly) collimated light source light. Optics may be used to provide (high) collimation (see also above).

Superluminescent diodes are known in the art. A superluminescent diode may be indicated as a semiconductor device which may be able to emit low-coherence light of a broad spectrum like a LED, while having a brightness in the order of a laser diode.

US2020192017 indicates for instance that “With current technology, a single SLED is capable of emitting over a bandwidth of, for example, at most 50-70 nm in the 800-900 nm wavelength range with sufficient spectral flatness and sufficient output power. In the visible range used for display applications, i.e. in the 450-650 nm wavelength range, a single SLED is capable of emitting over bandwidth of at most 10-30 nm with current technology. Those emission bandwidths are too small for a display or projector application which requires red (640 nm), green (520 nm) and blue (450 nm), i.e. RGB, emission”. Further, superluminescent diodes are amongst others described, in “Edge Emitting Laser Diodes and Superluminescent Diodes”, Szymon Stanczyk, Anna Kafar, Dario Schiavon, Stephen Najda, Thomas Slight, Piotr Perlin, Book Editor(s): Fabrizio Roccaforte, Mike Leszczynski, First published: 3 Aug. 2020) https://doi.org/10.1002/9783527825264.ch9 in chapter 9.3 superluminescent diodes. This book, and especially chapter 9.3, are herein incorporated by reference. Amongst others, it is indicated therein that the superluminescent diode (SLD) is an emitter, which combines the features of laser diodes and light-emitting diodes. SLD emitters utilize the stimulated emission, which means that these devices operate at current densities similar to those of laser diodes. The main difference between LDs and SLDs is that in the latter case, we design the device waveguide in a special way preventing the formation of a standing wave and lasing. Still, the presence of the waveguide ensures the emission of a high-quality light beam with high spatial coherence of the light, but the light is characterized by low time coherence at the same time” and “Currently, the most successful designs of nitride SLD are bent, curved, or tilted waveguide geometries as well as tilted facet geometries, whereas in all cases, the front end of the waveguide meets the device facet in an inclined way, as shown in FIG. 9.10. The inclined waveguide suppresses the reflection of light from the facet to the waveguide by directing it outside to the lossy unpumped area of the device chip”. Hence, an SLD may especially be a semiconductor light source, where the spontaneous emission light is amplified by stimulated emission in the active region of the device. Such emission is called “super luminescence”. Superluminescent diodes combine the high power and brightness of laser diodes with the low coherence of conventional light-emitting diodes. The low (temporal) coherence of the source has advantages that the speckle is significantly reduced or not visible, and the spectral distribution of emission is much broader compared to laser diodes, which can be better suited for lighting applications.

Especially, the first light generating device is configured to generate first device light. In embodiments, the first device light may comprise one or more of visible light and infrared radiation. Especially, the first device light may essentially consist of one or more of visible light and infrared radiation. Further, in embodiments the first light generating device comprises one or more of a superluminescent diode and a laser diode. Especially, at least 80% of the spectral power, even more especially at least 90%, such as at least 95% of the spectral power of the first device light may have a wavelength of at least 380 nm, like selected from the wavelength range of 380-1520 nm (see also below). For instance, a first light generating device emitting only at 600 nm has 100% of its spectral power in the wavelength range of 380-1520 nm.

The term “radiant flux” may especially refer to the radiant energy emitted per unit time (by the light generating device). Instead of the term “radiant flux”, also the terms “intensity” or “radian power” or “spectral power” may be applied. The term “radiant flux” may have as unit an energy, like especially Watts. The term “spectral power distribution” especially refers the power distribution of the light (especially in Watts) as function of the wavelength (especially in nanometers), especially in embodiments over the human visible wavelength range (380-780 nm). Especially, the term “spectral power distribution” may refer to a radiant flux per unit frequency or wavelength, often indicated in Watt/nm. Instead of the term “spectral power distribution” also the term “spectral flux” may be applied. Hence, instead of the phrase “controllable spectral power distribution”, also the phrase “controllable spectral flux” may be applied. The spectral flux may be indicated as power (Watt) per unit frequency or wavelength. Especially, herein the spectral flux is indicated as the radiant flux per unit wavelength (W/nm). Percentages of spectral power may especially to percentage of the spectral power in Watt.

It may be desirable to direct a substantial part of the first device light essentially only on the first converter material. For instance, at least 50%, like at least 60%, such as at least 70%, or even more especially at least about 80% or even at least about 90% of the first device light escaping from the first light generating device may be received by the first converter material without intermediate reflections. To this end, especially one or more lenses and/or other optics may be applied. For instance, focusing lenses may be applied. Would a pinhole be applied (see below), then especially (focusing) lenses may be applied. Hence, in specific embodiments the first light generating device may further comprise optics configured to provide focused first device light at the first converter material. For instance, the optics may comprise one or more focusing lenses.

Especially, the (optional) second light generating device is configured to generate visible second device light. The terms “visible”, “visible light” or “visible emission” and similar terms refer to light having one or more wavelengths in the range of about 380-780 nm. Herein, UV may especially refer to a wavelength selected from the range of 200-380 nm. In further specific embodiments, at least 80%, such as especially at least 90%, even more especially at least 95% of the spectral power of the second device light is within the 380-780 nm region. Hence, as indicated above, the second light generating device may be configured to generate visible second device light. For instance, a second light generating device emitting only in the wavelength range of 490-630 nm has 100% of its spectral power in the wavelength range of 380-780 nm.

Especially, the term “spectral power distribution” may refer to a radiant flux per unit frequency or wavelength, often indicated in Watt/nm. Instead of the term “spectral power distribution” also the term “spectral flux” may be applied. The term “radiant flux” may especially refer to the radiant energy emitted per unit time (by the light generating device). Instead of the term “radiant flux”, also the terms “intensity” or “radian power” may be applied. The term “radiant flux” may have as unit an energy, like especially Watts. Hence, instead of the phrase “controllable spectral power distribution”, also the phrase “controllable spectral flux” may be applied. The spectral flux may be indicated as power (Watt) per unit frequency or wavelength. Especially, herein the spectral flux is indicated as the radiant flux per unit wavelength (W/nm).

Upconversion may e.g. be based on upconverter luminescent materials or frequency doubling conversion materials. Both are herein indicated with the general term “converter material”. The term “luminescent material” may also refer to a plurality of different luminescent materials. Hence, the term “luminescent material” may in specific embodiments also refer to a luminescent material composition. The term “converter material” may also refer to a plurality of different converter materials. Hence, the term “converter material” may in specific embodiments also refer to a converter material composition.

The first device light may comprise one or more of visible light and infrared radiation. The first device light may be upconverted via the upconverter material. Hence, an upconversion of light having a wavelength of at least 380 nm may lead to first converter material light having a wavelength of smaller than 380 nm. Upconversion may be a frequency doubling (or wavelength halving), which may be done with frequency doubling materials like second harmonic generation crystals, such as e.g. BiBO (BiB₃O₆), Lithium iodate LiIO₃, BBO (β-BaB₂O₄), KH₂PO₄, etc., or based on luminescent materials, like e.g. based on the Yb³⁺—Er³⁺ couple (e.g. systems based on “addition de photons par transfer d'energie” (APTE), now generally known as energy transfer upconversion (ETU)), or via a two-step absorption process, such may be possible with e.g. Er³⁺ based systems, or via a cooperative sensitization process, such may be possible with the Yb³⁺—Tb³⁺ couple, or by a cooperative luminescence process, or via a two-photon excitation process, like e.g. possible with some Eu²⁺ based systems. Other couples may also be possible, e.g. Yb³⁺—Tm³⁺.

Another system may be based on Pr³⁺, and/or one or more of Ho³⁺, Tb³⁺, Tm³⁺, Er³⁺ such as described in US2010/0297206, which is herein incorporated by reference. For instance, in embodiments the material (which may also be indicated as “host material”) may be selected from the group consisting of NaLnF₄, LiLnF₄, KLnF₄, LnF₃, BaLn₂F₈, SrLn₂F₈, CaLn₂F₈, MgLn₂F₈; wherein Ln is (a) one or more of Pr³⁺, Yb³⁺, Ho³⁺, Tm³⁺, and Er³⁺; more especially wherein Ln³⁺ is Gd³⁺ and (b) one or more of Pr³⁺, Yb³⁺, Ho³⁺, Tm³⁺, and Er³⁺, wherein the material especially comprises at least a first lanthanide ion and a second lanthanide ion, different from the first lanthanide ion. For instance, in embodiments the material may be selected from the group consisting of NaLnF₄, LiLnF₄, NaLnF₄, LiLnF₄, KLnF₄, LnF₃, LiYF₄, KYF₄, BaLn₂F₈, SrLn₂F₈, CaLn₂F₈, MgLn₂F₈, BaLn₂F₈, SrLn₂F₈, CaLn₂F₈, or MgLn₂F₈, wherein Ln comprises (a) one or more of first lanthanide ions selected from the group of Gd³⁺, Pr³⁺, Tm³⁺, or Er³⁺ and (b) one or more second lanthanide ions selected from the group of Pr³⁺, Yb³⁺, Ho³⁺, Tm³⁺, Er³⁺, wherein the material comprises at least a first lanthanide ion and a second lanthanide ion, different from the first lanthanide ion. Especially, in embodiments Ln may comprise one or more of Lu and La, and at least one or more first lanthanide ions and one or more second lanthanide ions. Further, especially in embodiments Ln may comprise at least Gd, and optionally one or more of Lu and La, and at least one or more second lanthanide ions.

Further, it may also be possible to use semiconductor nanoparticles. For example, nanocrystals of perovskite CsPbBr₃ with molecular synthesizer showed more than 10% conversion efficiency emitting in the range 340-400 nm by excitation 440 nm may be obtained. For instance, it is referred to Wieghold S, Nienhaus L Correction: Engineering 3D perovskites for photon interconversion applications. PLOS ONE 15(4): e0232196. https://doi.org/10.1371/journal.pone.0232196.

Other up-conversion examples may e.g. be UV Pr³⁺ doped crystals like Lu₇O₆F₉:Pr³⁺ or Y₂SiO₅:Pr³⁺), or NaYF₄:Yb³⁺, Tm³⁺. Other examples can be found in Bright Infrared-to-Ultraviolet/Visible Upconversion in Small Alkaline Earth-Based Nanoparticles with Biocompatible CaF2 Shells—Fischer—2020—Angewandte Chemie International Edition-Wiley Online Library, which is herein incorporated by reference, and which describes NIR-to-UV/visible emission of sub-15 nm alkaline-earth rare-earth fluoride UCNPs (M₁₋ₓLnₓF₂₊ₓ, MLnF) with a CaF₂ shell. Different alkaline-earth host materials doped with Yb³⁺ and Tm³⁺, with alkaline-earth (M) spanning Ca, Sr, and Ba, MgSr, CaSr, CaBa, SrBa, and CaSrBa.

Further, it is referred to US2010/0297206, which is herein incorporated by reference. Further information about upconversion can also be found in e.g. G. Blasse et al., Luminescent Materials, Springer Verlag 1994, chapter 10.1. Hence, in embodiments the first converter material may comprise an upconverter luminescent material. Alternatively or additionally, the first converter material may comprises a frequency doubling (crystalline) material.

In embodiments, the light of a laser diode light source or superluminescent diode light source may be upconverted with a upconverter luminescent material. In embodiments, the light of a laser light source or superluminescent diode light source may be upconverted with a frequency doubling material.

In embodiments, the light of a laser crystal light source or may be upconverted with a upconverter luminescent material. In embodiments, the light of a laser crystal light source may be upconverted with a frequency doubling material. The laser crystal light source may be a combination of a laser diode (or superluminescent diode) light source and a lanthanide (or optionally transition metal) based material that can be pumped with the light source to get laser light out of the lanthanide (or optionally transition metal) based material.

Would the device light comprise infrared radiation, i.e. radiation having a wavelength of at least 780 nm, like e.g. selected from the range of 780-2000 nm, like 780-1300 nm, multiple upconversion steps may be necessary. Hence, especially the device light may comprise light having a wavelength selected from the range of 480-1520 nm, such as in embodiments selected from the range of 380-1140 nm, like selected from the range of 380-760 nm especially selected from the range of 380-560 nm or selected from the range of 760-1520 nm, like especially selected from the range of 760-1200 nm.

The light generating system may further comprise an optical element. Especially, the optical element may be used to reflect or transmit radiation. For instance, especially the optical element may be transmissive for the first device light. Hence, in specific embodiment the optical element may comprise a light window or part of a light window. For instance, the system may comprise a light exit window and the optical element may comprise part of the light exit window, or may comprise the entire light exit window.

Therefore, in embodiments the optical element may be configured in a light receiving relationship with the first light generating device. Especially, the optical element is transmissive for the first device light. In this way, at least part of the first device light may escape from the space defined by the chamber. The term transmissive may especially indicate that under perpendicular irradiation of the optical element with the first device light, the transmission of the first device light through the optical element may be at least 50%, more specially at least 70%, yet even more especially at least 85%, such as at least 90%. Hence, the phrase “wherein the optical element is transmissive for the first device light”, and similar phrases, may (thus) indicate that the optical element is at least partly transmissive for the first device light. Likewise, the phrase “reflective for the first converter material light”, and similar phrases may indicate at least partly reflective for the first converter material light. The phrase “the transmission of the first device light through the optical element may be at least x %”, and similar phrases, may especially indicate that averaged over the wavelengths of the spectral power distribution of the first device light, the transmission is x %. Hence, in the context of transmission and reflection, “at least partly” may in embodiments especially refer to wavelength dependent transmission values.

Downstream of the optical element, the first converter material layer may be configured.

The terms “upstream” and “downstream”, such as in the context of propagation of light, may especially relate to an arrangement of items or features relative to the propagation of the light from a light generating element (here the especially the . . . ), wherein relative to a first position within a beam of light from the light generating element, a second position in the beam of light closer to the light generating element (than the first position) is “upstream”, and a third position within the beam of light further away from the light generating element (than the first position) is “downstream”. For instance, instead of the term “light generating element” also the term “light generating means” may be applied.

The terms “radiationally coupled” or “optically coupled” may especially mean that (i) a light generating element, such as a light source, and (ii) another item or material, are associated with each other so that at least part of the radiation emitted by the light generating element is received by the item or material. In other words, the item or material is configured in a light-receiving relationship with the light generating element. At least part of the radiation of the light generating element will be received by the item or material. This may in embodiments be directly, such as the item or material in physical contact with the (light emitting surface of the) light generating element. This may in embodiments be via a medium, like air, a gas, or a liquid or solid light guiding material. In embodiments, also one or more optics, like a lens, a reflector, an optical filter, may be configured in the optical path between light generating element and item or material. The term “in a light-receiving relationship” does, as indicated above, not exclude the presence of intermediate optical elements, such as lenses, collimators, reflectors, dichroic mirrors, etc. In embodiments, the term “light-receiving relationship” and “downstream” may essentially be synonyms.

As indicated above, the first converter material is configured downstream of the optical element. The phrase “downstream of the optical element”, and similar phrases, may indicate downstream of at least part of the optical element (the first converter material may be configured). Further, the first converter material may especially be configured to convert at least part of the first device light transmitted by the optical element into first converter material light. In embodiments, the first light generating device and the first converter material are selected such that the first converter material light has spectral power at one or more wavelengths in the wavelength range of smaller than 380 nm. Hence, the first light generating device and the first converter material may be selected such that the first converter material light has spectral power in the UV wavelength range, especially in the wavelength range of 190-380 nm.

Further, as indicated above, the optical element may have a lower transmission for the first converter material light than for the first device light. For instance, this may be due to material intrinsic properties, like glasses or polymeric materials having a higher transmission for visible radiation than for (at least part of the) UV radiation. Alternatively or additionally, the optical element may comprise a color separation element (first color separation element) which may allow transmission from the first device light (through the optical element) into the first converter material, but which may attenuate transmission of the converter light in a direction from the first converter to the first light generating device. Hence, in embodiments the optical element may comprise a color separation element being transmissive for the first device light and reflective for the first converter material light. In embodiments, the color separation element may comprise a dichroic filter (or interference filter). For instance, the color separation element may be configured to transmit the first device light, especially laser light, while the color separation element may reflect the converter material light.

For instance, the optical element may comprise a first light transmissive layer, or at least part thereof (like a segment) (see also below).

The first light transmissive layer may in embodiments comprise a glass. In other embodiments, the first transmissive layer may comprise a polymeric material. In such embodiments, a downstream configured color separation element may be an option to further improve color separation. However, the first light transmissive layer as such may already have some color separation function as transmission of UV radiation may be smaller than the transmission of visible radiation. In yet further embodiments, the first transmissive layer may comprise quarts. In such embodiments, a downstream configured color separation element may be desirable to provide color separation.

The color separation element may comprise a dichroic filter. Instead of the dichroic filter, it may also be possible to use a reflector with a small opening, especially a pinhole. First device light may propagate through the small opening, and the converter material, downstream of the pinhole, may convert at least part of the light received by the converter material through the pinhole. Part of this first converter material light will propagate in the right direction without reflection. Part of this first converter material light will propagate in the right direction (only) after reflection at the reflector. A relatively small part of this first converter material light will propagate in wrong direction and escape via the small opening.

Hence, in embodiments the color separation element may comprise a pinhole, wherein the pinhole may be configured in a light receiving relationship with the first light generating device, and wherein the first converter material is configured downstream of the pinhole. Especially, the first converter material is configured downstream of the color separation element and (thus) also the pinhole.

The pinhole may have a hole cross-sectional area (Aₕ₁) and the color separation element may have a cross-sectional element area (A_{cse}) (not including the cross-sectional area of the pinhole), wherein Aₕ₁/A_{cse}≤0.2, such as Aₕ₁/A_{cse}≤0.1. For instance, in embodiments Aₕ₁/A_{cse}≥0.005.

The first device light may e.g. be focused on the pinhole or within the pinhole (or behind the pinhole in the converter material). Hence, in embodiments the first light generating device may (further) comprise optics configured to provide collimated or focused first device light at the first converter material. More especially, the first light generating device may (further) comprise optics configured to provide collimated or focused first device light at the first converter material via the pinhole. Especially, in such embodiments the first light generating device may comprise one or more of a superluminescent diode and a laser diode. Therefore, in specific embodiments at least 90% of the spectral power of the first light generating device light may enter the pinhole in such embodiments and at maximum 10% of the spectral power my not enter the pinhole, e.g. rays with too large angles relative to an optical axis of the first device light.

As indicated above, the system may comprise a light exit window. In embodiments, the light exit window comprises a first part, wherein the first part comprises at least part of the optical element. In specific embodiments, the first part is at least part of the optical element. Yet, in further specific embodiments the light exit window is at least part of the optical element. For instance, the light exit window may comprise a light transmissive material, e.g. selected from the group of glasses, polymeric materials, ceramics, and quartz. Such light transmissive material (or light exit window comprising light transmissive material) may in embodiments form a (light transmissive) envelope. Hence, the first part may comprise at least part of the first light transmissive layer.

Hence, in embodiments the invention (also) provides an envelope, such as for solid-state UV light disinfection.

In embodiments, the envelope may have the shape of a bulb. Hence, in specific embodiments the light generating system may comprise or be comprised by a retrofit lamp. In specific embodiments, the first light transmissive layer may have the shape of a bulb.

Hence, the light transmissive material may especially provide a first light transmissive layer. therefore, a light transmissive envelope may comprise the first light transmissive layer. In specific embodiments the light transmissive envelope may consist of the first light transmissive layer. Hence, in specific embodiments the light exit window may comprise a first light transmissive layer, especially over an entire cross-section of the light exit window; and downstream of at least part thereof the converter material may be configured. In specific embodiments, downstream of at least part of the light exit window, the color separation element may be configured.

Hence, in embodiments the system may further comprise a first light transmissive layer, wherein the optical element comprises at least part of the first light transmissive layer, wherein the (optional) color separation element, as defined herein, is configured between the first light transmissive layer and the first converter material.

In embodiments, downstream of the entire light exit window, the color separation element may be configured. In such embodiments, the entire light exit window may essentially be the optical element. It may also be that downstream of part of the light exit window; the color separation element may be configured. In such embodiments, the combination of (i) the part of the light exit window upstream of the color separation element and (ii) the color separation element may be defined as first part of the light exit window, and in embodiments another part of the light exit window, of which downstream no color separation element is configured, may be defined as second part of the light exit window. Hence, in specific embodiment the optical element may comprise a light window or part of a light window. For instance, the system may comprise a light exit window and the optical element may comprise part of the light exit window, or may comprise the entire light exit window:

The light exit window may in embodiments be part of a light chamber. In specific embodiments, the system may further comprise a light chamber. The light chamber may be configured in a light receiving relationship with the first light generating device. Hence, the first device light may be provided to or generated in the light chamber. Especially, the light chamber comprises a chamber wall. The chamber wall may comprise a first chamber wall part. In specific embodiments, the first chamber wall part comprises the optical element. Hence, in embodiments the system may further comprise a light chamber, wherein the light chamber is configured in a light receiving relationship with the first light generating device; wherein the light chamber comprises a chamber wall, wherein the chamber wall comprises a first chamber wall part, wherein the first chamber wall part comprises the optical element.

As indicated above, it may be desirable to direct a substantial part of the first device light essentially only on the first converter material. Hence, in embodiments a substantial part of the first device light may be received by the first chamber wall part. Would further wall parts be obtained, such wall parts may receive less first device light. For instance, at least 50%, like at least 60%, such as at least 70%, or even more especially at least about 80% or even at least about 90% of the (spectral power of the) first device light escaping from the first light generating device may be received by the first chamber wall part without intermediate reflections. To this end especially one or more lenses may be applied. For instance, focusing lenses may be applied. Would a pinhole be applied, see above or below; then especially (focusing) lenses may be applied. Hence, in specific embodiments the first light generating device further comprise optics configured to provide focused first device light at the first converter material.

The first device light may be generated in the light chamber or may be provided to the light chamber, e.g. via a light guide. Hence, the first device light may in embodiments be provided in the light chamber due to the presence of a light emitting surface, like a die, of the first light generating device in the light chamber. Alternatively or additionally, the first light generating device may be configured external from the light chamber, but the first device light is guided to the light chamber, e.g. via a light guide (having a light emitting surface in the light chamber). Especially, at least 90%, such as at least 95%, like 100% (of the spectral power) of the first device light that escapes from the first light generating device is provided in the light chamber.

Would a second light generating device be comprised by the system, especially at least 90%, such as at least 95%, like 100% (of the spectral power) of the second device light that escapes from the second light generating device is provided in the light chamber.

The light chamber may comprise a chamber wall, wherein at least part of the chamber wall is transmissive for the first device light and the optional second device light. The latter may be indicated as light exit window.

The light chamber may be defined by one or more reflective walls and the light exit window. The term “wall” may refer to essentially any face, like a side wall and a bottom wall. The latter may e.g. at least partly be provided by a printed circuit board. Light provided in the light chamber may therefore be reflected by the reflective walls or be transmitted by the light exit window. Especially, part of the light provided in the light chamber may be transmitted by the window: Light that is reflected by the light exit window or by reflective walls may be reused and may after one or more reflection reach the light exit window again, which may allow a further change to escape from the light chamber and be transmitted. Hence, the light chamber may at least partly be defined by the light exit window. The light chamber wall and the light exit window may define a (closed) chamber wherein at least part (especially a light emitting surface) of the first light generating device may be configured,

The light chamber may be defined by light reflective walls and by the light exit window. The reflective walls may be reflective for at least the first device light, and especially also for the optional second device light. Especially, the reflective walls are reflective for both the first device light and the second device light. When averaging over the internal surface area of the light chamber not taking into account the light exit window, the average reflectivity for the first device light-under perpendicular irradiation—may be at least 50%, even more especially at least 70%, yet even more especially at least 85%, such as at least 90%. Further, especially, when averaging over the internal surface area of the light chamber not taking into account the light exit window, the average reflectivity for also the optional second device light-under perpendicular irradiation—may be at least 50%, even more especially at least 70%, yet even more especially at least 85%, such as at least 90%. For instance, the walls may comprise an alumina coating or a Teflon coating, or may be reflective as such (see also below). Herein, reflectivity or transmission are especially defined under perpendicular irradiation. Note that this does not necessarily apply that the light for which the reflectivity or transmission of an element is defined, reaches the element (only) under perpendicular irradiation.

Further, the chamber wall may comprise a first chamber wall part. The first chamber wall part may especially comprise at least part of the optical element.

Especially, the first chamber wall part is the part of the chamber wall where first device light may escape from the chamber but is at least partly converted downstream thereof into light having another wavelength, especially light having one or more wavelengths in the UV.

Hence, in embodiments the first chamber wall part may comprises (i) an optical element or at least part thereof and (ii) a first converter material. Especially, the former may allow escape of at least part of the first device light in the light chamber from the light chamber. Further, especially the latter may allow conversion of at least part of the escaped light into first converter material light.

Hence, especially the first converter material may be configured downstream of the first light transmissive layer. Especially, the first converter material may be configured to convert at least part of the first device light transmitted by the optical element into first converter material light.

Especially, herein the first converter material is an upconversion material. Further, especially the first converter material is configured to generate radiation having spectral power in the UV wavelength range (especially of about 100-380 nm). Hence, in specific embodiments the first light generating device and the first converter material may be selected such that the first converter material light has spectral power at one or more wavelengths in the wavelength range of smaller than 380 nm.

Further, it may be desirable that first device light escapes via the first transmissive layer but that first converter material light does not substantially enter the light chamber via the first transmissive layer. This might lead to an undesirable light loss. Further, it is desirable that as much first converter material light emanates away from the light chamber. Then it may have e.g. a disinfection function in a space wherein the second device light may be provided (in an operational mode). Hence, especially in embodiments the first chamber wall part may comprise the color separation element, configured between optical element and the first converter material. More especially, the color separation element may be transmissive for the first device light and reflective for the first converter material light.

The phrase “color separation element may be transmissive for the first device light and reflective for the first converter material light” and may especially indicate that under perpendicular radiation of the intermediate element with the first device light, transmission of the first device light by the intermediate element is larger than the absorption or reflection of the first device light by the intermediate element. For instance, more than 50% of the first device light reaching the intermediate element perpendicularly may be transmitted, and less than 50% of the first device light reaching the intermediate element perpendicularly may be absorbed or reflected. Especially, more than 60% of the first device light reaching the intermediate element perpendicularly may be transmitted, and less than 40% of the first device light reaching the intermediate element perpendicularly may be absorbed or reflected. Further, this phrase may indicate that under perpendicular radiation of the intermediate element with the first converter material light, reflection of the first converter material light by the intermediate element is larger than the absorption or transmission of the first converter material light by the intermediate element. For instance, more than 50% of the first converter material light reaching the intermediate element perpendicularly may be reflected, and less than 50% of the first converter material light reaching the intermediate element perpendicularly may be absorbed or transmitted. Especially, more than 60% of the first converter material light reaching the intermediate element perpendicularly may be reflected, and less than 40% of the first converter material light reaching the intermediate element perpendicularly may be absorbed or transmitted.

In embodiments, the color separation element may be transmissive for a portion, such as a major portion, e.g. ≥70%, of the first device light and reflective for (a portion, such as a major portion e.g. ≥70%, of the first converter material light. Here, the percentages may again refer to a percentage of the spectral power distribution.

In embodiments, a transmission T_{cml} for the first converter material light and a transmission T_{fdl} for the first device light of the optical element may comply with T_{cml}≤0.5T_{fdl}, such as in embodiments T_{cml}≤0.1T_{fdl}, like especially in embodiments T_{cml}≤0.05T_{fdl}.

The first converter material may especially be provided as layer. Further, especially the optical element and the first converter material are layers, which may be comprised by a layer stack, wherein the first converter material is configured downstream of the first light transmissive layer.

The term “layer” may also refer to a plurality of stacked layers.

The color separation element may especially be provided as layer. Further, when the intermediate element is available, the optical element, the color separation element, and the first converter material may be layers, comprised by a layer stack, wherein the color separation element may (thus) be configured sandwiched between the first converter material and the first light transmissive layer.

As indicated above, the first chamber wall part may be the same as the at least part of the chamber wall that is transmissive for the second device light, the first chamber wall part and the light exit window may partly overlap, or the first chamber wall part and the light exit window do not overlap. In the former two embodiments, the first chamber wall part may be transmissive for the second device light. This may thus especially imply that the optical element, (ii) the first converter material; and an optional color separation element, are transmissive for the second device light.

The phrase “the first chamber wall part may be transmissive for the second device light”, and similar phrases, may especially indicate that under perpendicular radiation of the first chamber wall part with the second device light, transmission of the second device light by the first chamber wall part is larger than the absorption or reflection of the second device light by the first chamber wall part. For instance, more than 50% of the second device light reaching the first chamber wall part perpendicularly may be transmitted, and less than 50% of the second device light reaching the first chamber wall part perpendicularly may be absorbed or reflected. Especially, more than 60% of the second device light reaching the first chamber wall part perpendicularly may be transmitted, and less than 40% of the second device light reaching the first chamber wall part perpendicularly may be absorbed or reflected. Hence, in embodiments the first chamber wall part may be transmissive for the second device light.

However, it is herein not excluded that the first chamber wall part is reflective for second device light. In such embodiments, another part of the chamber wall may be transmissive for the second device light. For instance, the intermediate element may be reflective for both second device light and first converter material light.

A kind of hybrid solution may also be possible, wherein the color separation element as such may e.g. be reflective, but may comprise a small opening, like a pinhole. Through such pinhole first device light may escape from the light chamber, at least partly be transmitted through the light transmissive layer, and enter the luminescent material, where at least part of it may be converted into first converter material light. There the first converter material light may be radiated in many directions. A small part of it might be lost via the pinhole, but most of the first converter material light that would propagate in the direction of the color separation element will not meet the pinhole, and may be reflected at the color separation element. See further also above.

As indicated above, the system may comprise a first light generating device and optionally a second light generating device. Especially, the second light generating device may be configured to generate visible second device light, such as white light. In embodiments, the second light generating device may be configured to generate second device light of which the spectral power distribution is controllable. In embodiments, the second light generating device may comprise a (down converter) luminescent material (which may be indicated as second converter material: (down converter) luminescent materials are known in the art).

In embodiments, first part of the light exit window may be transmissive for the second device light. In other embodiments, when the system comprises a second part of the light exit window; the latter may be transmissive for the second device light, and the former may be transmissive for the second device light or may in specific embodiments be reflective for the second device light.

Hence, in embodiments wherein a light chamber may be applied, the light chamber may be configured in a light receiving relationship with the second light generating device. Further, especially the chamber wall may (thus be transmissive for the second device light (though the transmission may very over the part of the chamber wall that is transmissive for light. Hence, in embodiments the system may further comprise a second light generating device, wherein the second light generating device may be configured to generate visible second device light: wherein the light chamber may be configured in a light receiving relationship with the second light generating device; and wherein at least part of the chamber wall may be transmissive for the second device light. As indicated above, in specific embodiments the first chamber wall part may be transmissive for the second device light. In such embodiments, the (optional) color separation element and/or the first converter material, especially both the (optional) color separation element and the first converter material may be transmissive for the second device light.

As indicated above, the chamber wall may comprise a second chamber wall part. Especially, in such embodiments the second chamber wall part may be transmissive for the second device light. Therefore, in embodiments the chamber wall may comprise a second chamber wall part, and the second chamber wall part is transmissive for the second device light.

In specific embodiments, the second light generating device is configured to generate in an operational mode white light second device light (see also above). The term “white light” herein, is known to the person skilled in the art. It especially relates to light having a correlated color temperature (CCT) between about 1800 K and 20000 K, such as between 2000 and 20000 K, especially 2700-20000 K, for general lighting especially in the range of about 2700 K and 6500 K. In embodiments, for backlighting purposes the correlated color temperature (CCT) may especially be in the range of about 7000 K and 20000 K. Yet further, in embodiments the correlated color temperature (CCT) is especially within about 15 SDCM (standard deviation of color matching) from the BBL (black body locus), especially within about 10 SDCM from the BBL, even more especially within about 5 SDCM from the BBL.

As indicated above, the light exit window is transmissive for the first device light. Especially, this first device light may be transmitted through a first part of the light exit window. Further, in embodiments wherein the light exit window comprises a second part it may be desirable that the first device light does substantially not escape via this second part. To this end, the optical element may also comprise a part that is essentially not transmissive for the first device light but transmissive for the second device light and/or the first light generating device, and optional further optics, are configured such that essential all first device light that is transmitted through the optical element is transmitted through the first part, and not through other optional parts of the optical element. For instance, in embodiments part of an upstream side of the optical element may be coated with a reflector, that is reflective for the first device light.

Would a second light generating device be available, second device light of the second light generating device may be transmitted via (i) the first part of the optical element and the converter material configured downstream thereof and/or via (ii) an optional second part.

For recycling of light in a light chamber and/or for e.g. beam shaping applications, like spotlight applications, it may be desirable to use a reflector. Hence, in embodiments the chamber wall (see also above) may comprise a third chamber wall part, wherein the third chamber wall part is reflective for one or more of (i) the first device light and (ii) the second device light. Would in embodiments such third chamber wall part be available, especially the third chamber wall part may be reflective for the first device light.

The system may provide system light. In (first) operational modes, wherein only the first light generating device is applied, the system light may essentially consist of first converter material light. For instance, at least 90% of the spectral power of the system light may consist of the first converter material light, i.e. essentially all light has a wavelength of 380 nm or smaller, especially smaller than 380 nm. Hence, the optical element and the first converter material and the first light generating device may be configured such that essentially no first device light escapes from the system.

In yet further specific embodiments, a second optical element, more especially a second color separation element may be applied. The second color separation element may be configured downstream of the first converter material. The second optical element may allow transmission of the first converter material light (through the second optical element), but may attenuate transmission of the first device light (through the second optical element) in a direction from the first converter material to the external of system. Hence, in embodiments the second optical element may comprise a second color separation element being transmissive for the first converter material light and reflective (or absorbing) for the first device light. In embodiments, the second color separation element may comprise a dichroic filter (or interference filter). For instance, the second color separation element may be configured to transmit the first converter material light, while the second color separation element may reflect (and/or absorb) the (non-converted) first device light. In this way, essentially no (unconverted) first device light may escape from the system.

In embodiments, the second color separation element may be transmissive for a portion, such as a major portion, e.g. ≥70%, of the first converter material light and reflective for (a portion, such as a major portion e.g. ≥70%, of the first device light. Here, the percentages may again refer to a percentage of the spectral power distribution.

The system may further comprise a control system. The control system may control the first light generation device (and the optional second light generating device) in dependence of one or more of an input signal of a user interface, a sensor signal (of a sensor), and a timer. The term “timer” may refer to a clock and/or a predetermined time scheme.

In specific embodiments, the system may comprise a control system and a sensor, wherein the control system is configured to control a radiant flux of the first device light in dependence of the sensor (and optionally configured to control a radiant flux of the first device light in dependence of the sensor). For instance, in embodiments the sensor may be configured to detect people and to generate a related sensor signal, and wherein the control system is configured to control a radiant flux of the first device light in dependence of the sensor signal. Hence, in specific embodiments the control system is configured to control the first light generating device and the second light generating device in dependence of one or more of an input signal of a user interface, a sensor signal, and a timer.

In embodiments, the sensor may be selected from the group comprising a movement sensor, a presence sensor, a distance sensor, an ion sensor, a gas sensor, a volatile organic compound sensor, a pathogen sensor, an airflow sensor, a sound sensor, a temperature sensor, and a humidity sensor. A movement sensor may be used to sense people. A movement sensor may also be used to sense the number of people. A movement sensor may also be used to sense an activity level of the people (e.g. occupied or non-occupied working cubicle or fitness room). A presence sensor may be used to sense people. A presence sensor may also be used to sense the number of people. A presence sensor may also be used to sense an activity level of the people (e.g. occupied or non-occupied working cubicle or fitness room). A distance sensor may be used to sense one or more dimensions of a space for which the ionizer device is used. A distance sensor may also be used to sense distances between people. The ion sensor may comprise a positive ion sensor. Additionally or alternatively, the ion sensor may comprise a negative ion sensor. The ion sensor may be used to sense the effect of the ionizer device (the more ions, the better the air treatment may be). A gas sensor may be used to sense gas one or more gas components. The gas sensor may be used to sense whether ventilation is sufficient or insufficient. The gas sensor may e.g. (thus) also be used to sense the number of people and/or an activity level of the people. A volatile organic compound (VOG) sensor may be used to sense one or more volatile organic compounds. The VOG sensor may be used to sense whether ventilation is sufficient or insufficient. The VOG sensor may e.g. (thus) also be used to sense the number of people and/or an activity level of the people. The pathogen sensor may comprise a sensor for one or more of bacteria, viruses, and spores. The pathogen sensor may be used to sense whether ventilation is sufficient or insufficient. The pathogen sensor may e.g. (thus) also be used to sense the number of people and/or an activity level of the people. An airflow sensor may be used to sense an airflow. The airflow sensor may be used to sense whether ventilation is sufficient or insufficient. The airflow sensor may e.g. (thus) also be used to sense the number of people and/or an activity level of the people. A sound sensor may be used to sense sound. The sound sensor may be used to sense whether ventilation is sufficient or insufficient. The sound sensor may e.g. (thus) also be used to sense the number of people and/or an activity level of the people. The temperature sensor may be used to sense temperature. On the basis thereon, it may be determined whether pathogens may be more detrimental or less detrimental. The humidity sensor may be used to sense (air) humidity. On the basis thereon, it may be determined whether pathogens may be more detrimental or less detrimental (as there seems to be a relation between humidity and transferability of e.g. airborne pathogens).

Hence, the system may especially be used to provide first converter material light in a space, like a room (see also below). The first converter material light may essentially consist of UV radiation, like e.g. UV-C radiation.

In yet a further aspect, the invention also provides a method for one or more of (i) treating a gas or a surface in a space (external from the light generating system according to any one of the preceding claims), and (ii) providing light to the space, the method comprising providing the first converter material light with the radiation generating system as defined herein.

The term “space” may for instance relate to a (part of) hospitality area, such as a restaurant, a hotel, a clinic, or a hospital, etc., The term “space” may also relate to (a part of) an office, a department store, a warehouse, a cinema, a church, a theatre, a library, etc. However, the term “space” may also relate to (a part of) a working space in a vehicle, such as a cabin of a truck, a cabin of an air plane, a cabin of a vessel (ship), a cabin of a car, a cabin of a crane, a cabin of an engineering vehicle like a tractor, etc., The term “space” may also relate to (a part of) a working space, such as an office, a (production) plant, a power plant (like a nuclear power plant, a gas power plant, a coal power plant, etc.), etc. For instance, the term “space” may also relate to a control room, a security room, etc., Especially, the term “space” may herein refer to an indoor space. In yet other embodiments, the term “space” may also relate to a toilet room or bathroom. In yet other embodiments, the term “space” may also relate to an elevator. In embodiments, the term “space” may also refer to a conference room, a school room, an indoor hallway, an indoor corridor, an indoor space in an elderly home, an indoor space in a nursing home, etc. In embodiments, the term “space” may refer to an indoor sport space, like a gym, a gymnastics hall, in indoor ball sport space, a ballet room, a swimming pool, a changing room, etc. In embodiments, the term “space” may refer to an (indoor) bar, an (indoor) disco, etc.

In yet a further aspect, the invention also provides a lamp or a luminaire comprising the light generating system as defined herein. The luminaire may further comprise a housing, optical elements, louvres, etc. etc. . . . . The lamp or luminaire may further comprise a housing enclosing the light generating system. The lamp or luminaire may comprise a light window in the housing or a housing opening, through which the system light may escape from the housing. In specific embodiments, the lamp may be a retrofit lamp. In yet a further aspect, the invention also provides a projection device comprising the light generating system as defined herein. Especially, a projection device or “projector” or “image projector” may be an optical device that projects an image (or moving images) onto a surface, such as e.g. a projection screen. The projection device may include one or more light generating systems such as described herein. Hence, in an aspect the invention also provides a light generating device selected from the group of a lamp, a luminaire, a projector device, a disinfection device, a photochemical reactor, and an optical wireless communication device, comprising the light generating system as defined herein. The light generating device may comprise a housing or a carrier, configured to house or support, one or more elements of the light generating system. For instance, in embodiments the light generating device may comprise a housing or a carrier, configured to house or support the first light generating device and the optional second light generating device.

The light generating system may be part of or may be applied in e.g. office lighting systems, household application systems, shop lighting systems, home lighting systems, accent lighting systems, spot lighting systems, theater lighting systems, fiber-optics application systems, projection systems, self-lit display systems, pixelated display systems, segmented display systems, warning sign systems, medical lighting application systems, indicator sign systems, decorative lighting systems, portable systems, automotive applications, (outdoor) road lighting systems, urban lighting systems, green house lighting systems, horticulture lighting, digital projection, or LCD backlighting. The light generating system (or luminaire) may be part of or may be applied in e.g. optical communication systems or disinfection systems.

The terms “light” and “radiation” are herein interchangeably used, unless clear from the context that the term “light” only refers to visible light. The terms “light” and “radiation” may thus refer to UV radiation, visible light, and IR radiation. In specific embodiments, especially for lighting applications, the terms “light” and “radiation” refer to (at least) visible light.

The terms “violet light” or “violet emission” especially relates to light having a wavelength in the range of about 380-440 nm. The terms “blue light” or “blue emission” especially relates to light having a wavelength in the range of about 440-495 nm (including some violet and cyan hues). The terms “green light” or “green emission” especially relate to light having a wavelength in the range of about 495-570 nm. The terms “yellow light” or “yellow emission” especially relate to light having a wavelength in the range of about 570-590 nm. The terms “orange light” or “orange emission” especially relate to light having a wavelength in the range of about 590-620 nm. The terms “red light” or “red emission” especially relate to light having a wavelength in the range of about 620-780 nm. The term “pink light” or “pink emission” refers to light having a blue and a red component. The term “cyan” may refer to one or more wavelengths selected from the range of about 490-520 nm. The term “amber” may refer to one or more wavelengths selected from the range of about 585-605 nm, such as about 590-600 nm. The phrase “light having one or more wavelengths in a wavelength range” and similar phrases may especially indicate that the indicated light (or radiation) has a spectral power distribution with at least intensity or intensities at these one or more wavelengths in the indicate wavelength range. For instance, a blue emitting solid state light source will have a spectral power distribution with intensities at one or more wavelengths in the 440-495 nm wavelength range.

Herein, IR (infrared) may especially refer to radiation having a wavelength selected from the range of 780-3000 nm, such as 780-2000 nm, e.g. a wavelength up to about 1500 nm, like a wavelength of at least 900 nm, though in specific embodiments other wavelengths may also be possible. Hence, the term IR may herein refer to one or more of near infrared (NIR (or IR-A)) and short-wavelength infrared (SWIR (or IR-B)), especially NIR.

The term “controlling” and similar terms especially refer at least to determining the behavior or supervising the running of an element. Hence, herein “controlling” and similar terms may e.g. refer to imposing behavior to the element (determining the behavior or supervising the running of an element), etc., such as e.g. measuring, displaying, actuating, opening, shifting, changing temperature, etc., Beyond that, the term “controlling” and similar terms may additionally include monitoring. Hence, the term “controlling” and similar terms may include imposing behavior on an element and also imposing behavior on an element and monitoring the element. The controlling of the element can be done with a control system, which may also be indicated as “controller”. The control system and the element may thus at least temporarily, or permanently, functionally be coupled. The element may comprise the control system. In embodiments, the control system and element may not be physically coupled. Control can be done via wired and/or wireless control. The term “control system” may also refer to a plurality of different control systems, which especially are functionally coupled, and of which e.g. one control system may be a master control system and one or more others may be slave control systems. A control system may comprise or may be functionally coupled to a user interface.

The control system may also be configured to receive and execute instructions form a remote control. In embodiments, the control system may be controlled via an App on a device, such as a portable device, like a Smartphone or I-phone, a tablet, etc. The device is thus not necessarily coupled to the lighting system, but may be (temporarily) functionally coupled to the lighting system.

Hence, in embodiments the control system may (also) be configured to be controlled by an App on a remote device. In such embodiments the control system of the lighting system may be a slave control system or control in a slave mode. For instance, the lighting system may be identifiable with a code, especially a unique code for the respective lighting system. The control system of the lighting system may be configured to be controlled by an external control system which has access to the lighting system on the basis of knowledge (input by a user interface of with an optical sensor (e.g. QR code reader) of the (unique) code. The lighting system may also comprise means for communicating with other systems or devices, such as on the basis of Bluetooth, WIFI, LiFi, ZigBee, BLE or WiMAX, or another wireless technology.

The system, or apparatus, or device may execute an action in a “mode” or “operation mode” or “mode of operation” or “operational mode”. The term “operational mode may also be indicated as “controlling mode”. Likewise, in a method an action or stage, or step may be executed in a “mode” or “operation mode” or “mode of operation” or “operational mode”. This does not exclude that the system, or apparatus, or device may also be adapted for providing another controlling mode, or a plurality of other controlling modes. Likewise, this may not exclude that before executing the mode and/or after executing the mode one or more other modes may be executed.

However, in embodiments a control system may be available, that is adapted to provide at least the controlling mode. Would other modes be available, the choice of such modes may especially be executed via a user interface, though other options, like executing a mode in dependence of a sensor signal or a (time) scheme, may also be possible. The operation mode may in embodiments also refer to a system, or apparatus, or device, that can only operate in a single operation mode (i.e. “on”, without further tunability).

Hence, in embodiments, the control system may control in dependence of one or more of an input signal of a user interface, a sensor signal (of a sensor), and a timer. The term “timer” may refer to a clock and/or a predetermined time scheme.

### BRIEF DESCRIPTION OF THE DRAWINGS

Embodiments of the invention will now be described, by way of example only, with reference to the accompanying schematic drawings in which corresponding reference symbols indicate corresponding parts, and in which:

FIGS. 1a-1c schematically depict some embodiments:

FIG. 2 schematically depict some further embodiments. The schematic drawings are not necessarily to scale.

### DETAILED DESCRIPTION OF THE EMBODIMENTS

It is desired to protect yourself and others from the spread of bacteria and viruses such as influenza or against the outbreak of novel viruses like the recent COVID-19. UV light can be used for disinfection. However, because UV light may be absorbed and/or reflected by glass the solid state lighting emitters may not be enveloped by a glass envelope/protected by glass exit window. Hence, more difficult to handle and shape quarts may be needed. Amongst others, it is herein proposed to use a solid-state light emitter based system comprising one or more LEDs and/or lasers which are shielded by a glass or polymer) envelope or exit window. The envelope or exit window may be UV light absorbing and/or reflecting UV light. The solid state light emitter may especially be configured to emit light in in the violet range, such as having wavelength selected from the range of 380-420 nm, and/or further in the visible, such as up to about 780 nm, or even beyond 780 nm, like in the IR, such as wavelengths of at least 800 nm. Downstream of the envelope or exit window an up-conversion material, like an upconversion phosphor, may be arranged for converting violet and/or visible and/or IR light into UV light (especially <380 nm).

For this purpose, up-convention materials can be used. Lanthanide-doped nanoparticles are nanocrystals of a transparent material (more often the fluorides NaYF₄, NaGdF₄, LiYF₄, YF₃, CaF₂ or oxides such as Gd₂O₃) doped with certain amounts of lanthanide ions. The most common lanthanide ions used in photon up-conversion are the pairs erbium-ytterbium (Er³⁺, Yb³⁺) or thulium-ytterbium (Tm³⁺, Yb³⁺). In such combinations ytterbium ions may be added as antennas, to absorb light at around 980 nm and transfer it to the upconverter ion. If this ion is erbium, then a characteristic green and red emission is observed, while when the upconverter ion is thulium, thulium the emission includes near-ultraviolet, blue and red light. As the absorption spectrum of these materials are relatively narrow; they need to be excited by lasers. Up-conversion luminescence spectra of NaYF₄ crystals doped with different amounts of Yb³⁺ ions (29%, 49%, 69%, and 99%), under 980 nm NIR excitation. It is also possible to use semiconductor nanoparticles. For example, nanocrystals of perovskite CsPbBr₃ with molecular synthesizer showed more than 10% conversion efficiency emitting in the range 340-400 nm by excitation 440 nm. Up conversion efficiency may be related to the intensity and intensity excitation light needs to be higher than 2 W/cm². It may therefore be useful to concentrate light on a small area for efficient conversion of visible and IR light to UV light as shown below. The obtained effect may be a glass or polymer based envelope or exit window for solid-state UV light disinfection without showing absorption of the UV light.

In embodiments, a semi-reflective element e.g. a diffuser may be arranged between the envelope/exit window and the up-conversion phosphor. In case a laser or focused LEDs are used a reflector with a pinhole may be used. Other optical components for spreading the UV light may be used as well. The lighting device may be a luminaire (comprising a mounting means for mounting the luminaire to a wall or ceiling) or a lamp (comprising a cap, a driver and optionally a controller and an antenna).

Frequency doubling (FD) may also be used. A FD crystal may be arranged downstream the glass exit window or envelope, e.g. in a MR16 lamp, to convert visible to UV light.

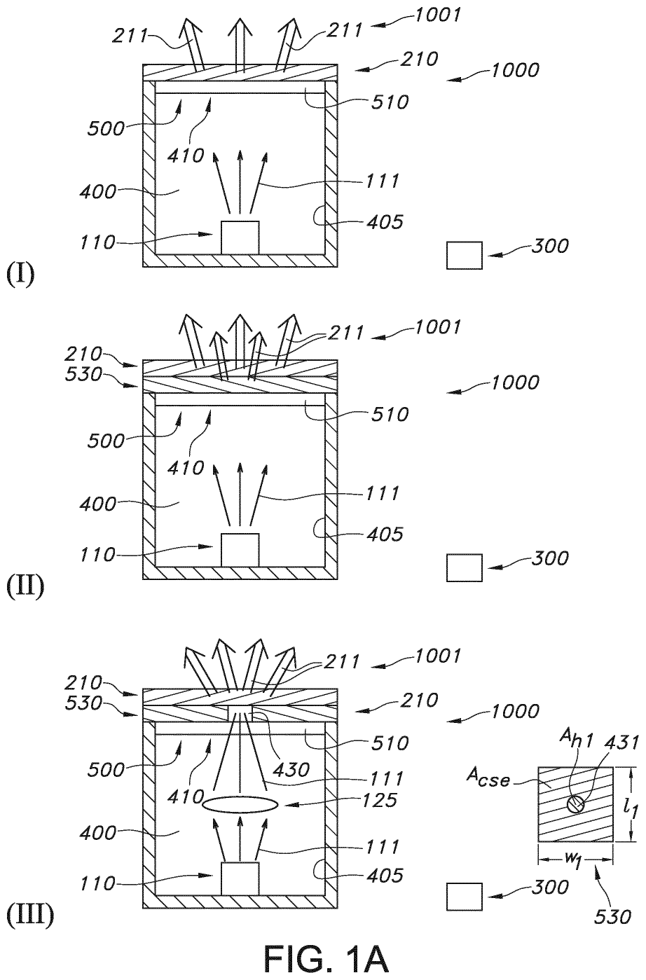

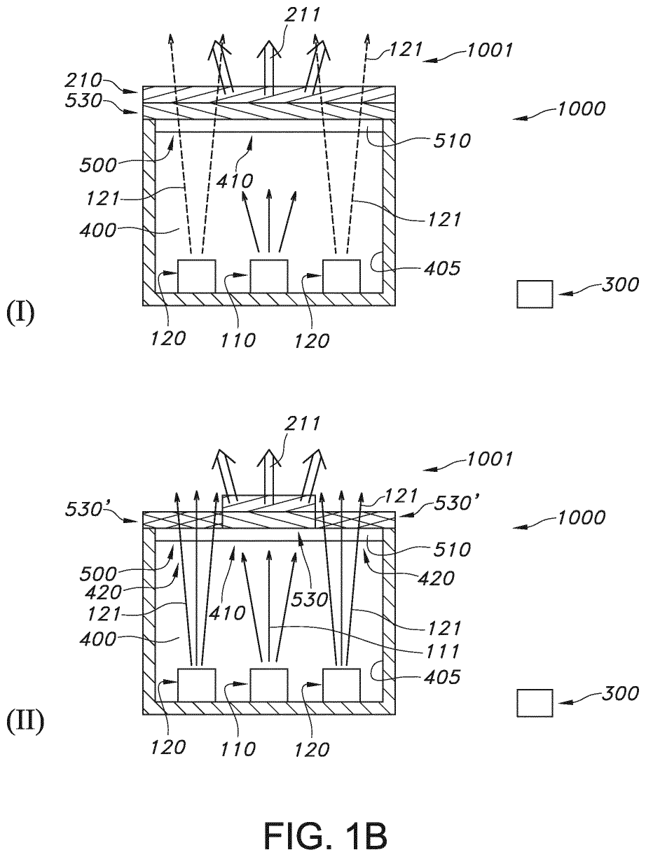

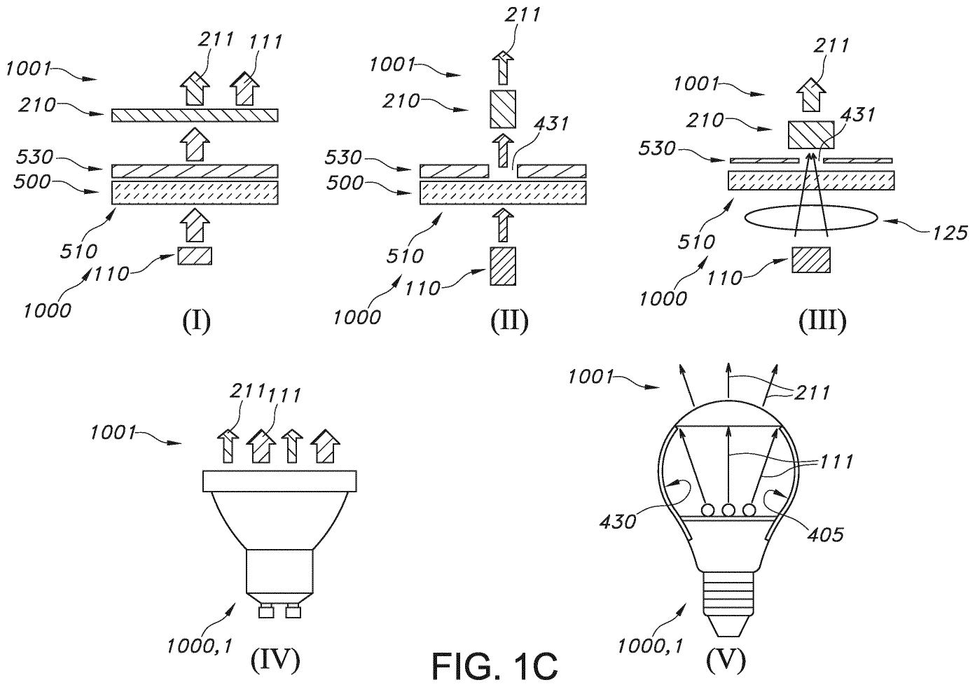

FIG. 1a schematically depicts three embodiments of a light generating system 1000 may comprise a first light generating device 110, an optical element 500, and a first converter material 210. The first light generating device 110 may be configured to generate first device light 111. The first device light 111 may comprise one or more of visible light and infrared radiation. The optical element 500 may be configured in a light receiving relationship with the first light generating device 110. The optical element 500 may especially be transmissive for the first device light 111. The first converter material 210 may be configured downstream of the optical element 500. The first converter material 210 may be configured to convert at least part of the first device light 111 transmitted by the optical element 500 into first converter material light 211. The first light generating device 110 and the first converter material 210 may be selected such that the first converter material light 211 may have spectral power at one or more wavelengths in the wavelength range of smaller than 380 nm. The optical element 500 may have a lower transmission for the first converter material light 211 than for the first device light 111. In specific embodiments, the optical element 500 may essentially not be transmissive for the first converter material light 211. The first light generating device 110 may comprise one or more of a superluminescent diode and a laser diode.

In embodiments, the first converter material 210 may comprise an upconverter luminescent material. In (other) embodiments, the first converter material 210 may comprise a frequency doubling (crystalline) material.

The light generating system 1000 may further comprise a first light transmissive layer 510. The optical element 500 may comprise at least part of the first light transmissive layer 510.

The light generating system 1000 may further comprise a control system 300. The control system 300 may be configured to control the first light generating device 110 and the second light generating device 120 in dependence of one or more of an input signal of a user interface, a sensor signal, and a timer.

The light generating system 1000 may further comprise a light chamber 400. The light chamber 400 may be configured in a light receiving relationship with the first light generating device 110. The light chamber 400 may comprise a chamber wall 405, wherein the chamber wall 405 may comprise a first chamber wall part 410. The first chamber wall part 410 may comprise the optical element 500.

Reference 1001 refers to light escaping from the system 1000. In an operational mode, the light 1001 escaping from the system comprises first converter material light 211.

Referring to embodiments II and III of FIG. 1a, the optical element 500 may comprise a color separation element 530 being transmissive for the first device light 111 and reflective for the first converter material light 211. In embodiments, the color separation element 530 may be configured between the first light transmissive layer 510 and the first converter material 210.

Referring to embodiment II, the color separation element 530 may e.g. comprise a dichroic filter, like a dichroic layer.

Referring to embodiment III of FIG. 1a, the color separation element 530 may comprise a pinhole 431. The pinhole 431 may be configured in a light receiving relationship with the first light generating device 110. Especially, the first converter material 210 may be configured downstream of the pinhole 431. Further, the first light generating device 110 may comprise optics 125 configured to provide collimated or focused first device light 111 at the first converter material 210.

In the drawing of embodiment III of FIG. 1a, also an insert is shown, which shows a cross-sectional vies of the color separation element 530 and the pinhole 431. The pinhole may have a hole cross-sectional area (Aₕ₁) and the color separation element may have a cross-sectional element area (A_{cse}) (not including the cross-sectional area of the pinhole), wherein Aₕ₁/A_{cse}≤0.2, such as Aₕ₁/A_{cse}≤0.1. References l1 and w1 may indicate a length and a width of the color separation element 530. Hence, especially l1*w1=Aₕ₁+A_{cse}. FIG. 1b schematically depict some further embodiments, especially as variations on embodiment II of FIG. 1a. However, similar variants may be provided on the bases if embodiment I or III of FIG. 1a.

Referring to embodiments I and II of FIG. 1b, the light generating system 1000 may further comprise a second light generating device 120. The second light generating device 120 may be configured to generate visible second device light 121. In embodiments, the light chamber 400 may be configured in a light receiving relationship with the second light generating device 120. Especially, at least part of the chamber wall 405 may be transmissive for the second device light 121. This part may in embodiments be the first part or the first chamber wall part 410, though other embodiments may also be possible. Hence, in specific embodiments the first chamber wall part 410 is transmissive for the second device light 121.

Reference 1001 refers to light escaping from the system 1000. In an operational mode, the light 1001 escaping from the system may comprise first converter material light 211. In another operational mode, the light 1001 escaping from the system may comprise second device light 121. In yet another operational mode, the light 1001 escaping from the system may comprise first converter material light 211 and second device light 121.

Referring to embodiment II, the chamber wall 405 may comprise a second chamber wall part 420. The second chamber wall part 420 may be transmissive for the second device light 121. The second wall part may optionally comprise other color separation elements, indicated with references 530″.

In specific embodiments, the second light generating device 120 may be configured to generate in an operational mode white light second device light 121.

As schematically depicted, the optical element may comprise a first light transmissive layer, or at least part thereof (like a segment).

FIG. 1c schematically depict some aspects, illustrated with embodiments I-V. embodiment I shows a variant wherein the optical element 500 may comprise a light transmissive layer 510 and a dichroic mirror or reflector as color separation element 530. Alternatively or in addition, a diffuser may be used. Embodiment II shows an embodiment wherein the optical element 500 may comprise a light transmissive layer 510 and a reflector comprising a pinhole 430 as color separation element 530. Embodiment III is essentially the same as embodiment II, but now with further optics 125 for focusing the beam of first light device light 111. Embodiments IV and V schematically depict specific embodiments of lamps 1. Hence, a lamp 1 may comprise the light generating system. Embodiment V schematically depicts an embodiment of (a lamp 1 comprising) the light generating system 1000, wherein the chamber wall 405 may comprise a third chamber wall part 430. The third chamber wall part 430 may be reflective for one or more of the first device light 111 and the second device light 121.

Not depicted in FIGS. 1a and 1b, are possible further optics downstream of the first converter material 210. For instance, downstream thereof one or more lenses and/or one or more collimators may be configured.

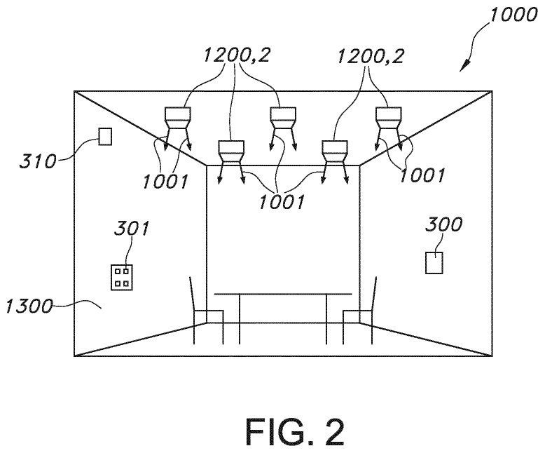

FIG. 2 schematically depicts an embodiment of a luminaire 2 comprising the light generating system 1000 as described above. Reference 301 indicates a user interface which may be functionally coupled with the control system 300 comprised by or functionally coupled to the light generating system 1000. As indicated above, FIG. 1c embodiments IV and V schematically depicts an embodiment of lamp 1 comprising the light generating system 1000.

Hence, the invention also provides a lighting device 1200 selected from the group of a lamp 1, a luminaire 2, a projector device, a disinfection device, a photochemical reactor, and an optical wireless communication device, comprising the light generating system 1000. In embodiments, such lighting device may be a lamp 1, a luminaire 2, a projector device, a disinfection device, or an optical wireless communication device.

The light generating system 1000 may further comprise a control system 300 and a sensor 310, wherein the control system 300 may be configured to control a radiant flux of the first device light 111 in dependence of the sensor 310. In embodiments, the sensor 310 may be configured to detect people and to generate a related sensor signal, and the control system (300) may be configured to control a radiant flux of the first device light (111) in dependence of the sensor signal.

The invention also provides a method for one or more of treating a gas or a surface in a space 1300 (external from the light generating system 1000, and providing light to the space 1300, the method may comprise providing first converter material light 211 with the radiation generating system 1000 to the gas or the surface.

The term “plurality” refers to two or more. The terms “substantially” or “essentially” herein, and similar terms, will be understood by the person skilled in the art. The terms “substantially” or “essentially” may also include embodiments with “entirely”, “completely”, “all”, etc. Hence, in embodiments the adjective substantially or essentially may also be removed. Where applicable, the term “substantially” or the term “essentially” may also relate to 90% or higher, such as 95% or higher, especially 99% or higher, even more especially 99.5% or higher, including 100%. The term “comprise” also includes embodiments wherein the term “comprises” means “consists of”. The term “and/or” especially relates to one or more of the items mentioned before and after “and/or”. For instance, a phrase “item 1 and/or item 2” and similar phrases may relate to one or more of item 1 and item 2. The term “comprising” may in an embodiment refer to “consisting of” but may in another embodiment also refer to “containing at least the defined species and optionally one or more other species”.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The devices, apparatus, or systems may herein amongst others be described during operation. As will be clear to the person skilled in the art, the invention is not limited to methods of operation, or devices, apparatus, or systems in operation.

It should be noted that the above-mentioned embodiments illustrate rather than limit the invention, and that those skilled in the art will be able to design many alternative embodiments without departing from the scope of the appended claims.

In the claims, any reference signs placed between parentheses shall not be construed as limiting the claim.

Use of the verb “to comprise” and its conjugations does not exclude the presence of elements or steps other than those stated in a claim. Unless the context clearly requires otherwise, throughout the description and the claims, the words “comprise”, “comprising”, and the like are to be construed in an inclusive sense as opposed to an exclusive or exhaustive sense: that is to say, in the sense of “including, but not limited to”. The article “a” or “an” preceding an element does not exclude the presence of a plurality of such elements.

The invention may be implemented by means of hardware comprising several distinct elements, and by means of a suitably programmed computer. In a device claim, or an apparatus claim, or a system claim, enumerating several means, several of these means may be embodied by one and the same item of hardware. The mere fact that certain measures are recited in mutually different dependent claims does not indicate that a combination of these measures cannot be used to advantage. In yet a further aspect, the invention (thus) provides a software product, which, when running on a computer is capable of bringing about (one or more embodiments of) the method as described herein.

The invention also provides a control system that may control the device, apparatus, or system, or that may execute the herein described method or process. Yet further, the invention also provides a computer program product, when running on a computer which is functionally coupled to or comprised by the device, apparatus, or system, controls one or more controllable elements of such device, apparatus, or system.

The invention further applies to a device, apparatus, or system comprising one or more of the characterizing features described in the description and/or shown in the attached drawings. The invention further pertains to a method or process comprising one or more of the characterizing features described in the description and/or shown in the attached drawings.

The various aspects discussed in this patent can be combined in order to provide additional advantages. Further, the person skilled in the art will understand that embodiments can be combined, and that also more than two embodiments can be combined. Furthermore, some of the features can form the basis for one or more divisional applications.

1. A light generating system comprising (i) a first light generating device, an optical element, and a first converter material, wherein:
the first light generating device is configured to generate first device light, wherein the first device light comprises one or more of visible light and infrared radiation;
the optical element is configured in a light receiving relationship with the first light generating device; wherein the optical element is transmissive for the first device light;
the first converter material is configured downstream of the optical element; wherein the first converter material is configured to convert at least part of the first device light transmitted by the optical element into first converter material light; wherein the first light generating device and the first converter material are selected such that the first converter material light has spectral power at one or more wavelengths in the wavelength range of smaller than 380 nm;
the optical element has a lower transmission for the first converter material light than for the first device light; and
the optical element comprises a color separation element being transmissive for the first device light and reflective for the first converter material light.

2. The light generating system according to claim 1, wherein the color separation element comprises a pinhole, wherein the pinhole is configured in a light receiving relationship with the first light generating device, and wherein the first converter material is configured downstream of the pinhole.

3. The light generating system according to claim 1, wherein the color separation element comprises a dichroic mirror.

4. The light generating system according to claim 1, wherein the first light generating device comprises one or more of a superluminescent diode and a laser diode; wherein the first light generating device further comprise optics configured to provide collimated or focused first device light at the first converter material.

5. The light generating system according to claim 1, wherein the first converter material comprises an upconverter luminescent material.

6. The light generating system according to claim 1, wherein the first converter material comprises a frequency doubling material.

7. The light generating system according to claim 1, further comprising a light chamber, wherein the light chamber is configured in a light receiving relationship with the first light generating device; wherein the light chamber comprises a chamber wall, wherein the chamber wall comprises a first chamber wall part, wherein the first chamber wall part comprises the optical element.

8. The light generating system according to claim 7, further comprising a second light generating device, wherein the second light generating device is configured to generate visible second device light; wherein the light chamber is configured in a light receiving relationship with the second light generating device; and wherein at least part of the chamber wall is transmissive for the second device light.

9. The light generating system according to claim 8, wherein the first chamber wall part is transmissive for the second device light.

10. The light generating system according to claim 7, wherein the chamber wall comprises a second chamber wall part, wherein the second chamber wall part is transmissive for the second device light.

11. The light generating system according to claim 7, wherein the second light generating device is configured to generate in an operational mode white light second device light.

12. The light generating system according to claim 6, wherein the chamber wall comprises a third chamber wall part, wherein the third chamber wall part is reflective for one or more of (i) the first device light and (ii) the second device light.

13. The light generating system according to claim 1, further comprising a first light transmissive layer, wherein the optical element comprises at least part of the first light transmissive layer, wherein the color separation element is configured between the first light transmissive layer and the first converter material.

14. A method for one or more of (i) treating a gas or a surface in a space, and (ii) providing light to the space, the method comprising providing the first converter material light with the radiation generating system according to claim 1 to the gas or the surface.

15. A lighting device selected from the group of a lamp, a luminaire, a projector device, a disinfection device, a photochemical reactor, and an optical wireless communication device, comprising the light generating system according to claim 1.
